(19) United States
(12) Patent Application Publication
(10) Pub. No.: US 2017/0165217 A1
(43) Pub. Date: Jun. 15, 2017

(54) LIQUID DOSAGE FORMS OF ISOTRETINOIN

(71) Applicant: Sun Pharmaceutical Industries Limited, Mumbai (IN)

(72) Inventors: Sanjay Kumar MOTWANI, Bhopal (IN); Shashikanth P. ISLOOR, Shimoga (IN); Vinod Kumar ARORA, Gurgaon (IN)

(21) Appl. No.: 15/444,491

(22) Filed: Feb. 28, 2017

Related U.S. Application Data
(63) Continuation of application No. 13/320,164, filed on Jun. 25, 2012.

(30) Foreign Application Priority Data
May 20, 2009 (IN) 1039/DEL/2009

(51) Int. Cl. A61K 31/203; A61K 9/00; A61K 47/12; A61K 9/08

(52) U.S. Cl. CPC A61K 31/203; A61K 9/08; A61K 9/0053; A61K 47/12

(57) ABSTRACT

The present invention relates to a pharmaceutical solution comprising isotretinoin or salts thereof. The present invention further relates to the processes for preparing such compositions.

### TECHNICAL FIELD OF THE INVENTION

The present invention relates to a pharmaceutical solution comprising isotretinoin or a pharmaceutically acceptable salt of isotretinoin and a process for the preparation thereof.

### BACKGROUND OF THE INVENTION

Isotretinoin is a retinoid, approved for the treatment of severe recalcitrant nodular acne. Chemically, isotretinoin is 13-cis-retinoic acid and is related to both retinoic acid and retinol (vitamin A).

Presently isotretinoin is marketed by Hoffman La Roche under the brand name Accutane®. This product comprises a suspension of isotretinoin filled in soft gelatin capsules.

U.S. Pat. No. 4,322,438, assigned to Hoffman-La Roche, discloses a method of treating nodulocystic and conglobate acne in humans by oral administration of 13-cis-retinoic acid in amounts and for periods of time which afford an effectively complete remission from the condition even after administration of the compound ceases.

PCT Publication No. WO 00/25772, filed by Hoffman-La Roche, relates to soft gel capsules of isotretinoin having improved bioavailability. This application discloses that the currently marketed Accutane® formulation of isotretinoin has a mean particle size of 100 μm and has a bioavailability of only about 20%. Therefore, it discloses a process of further reducing the particle size of isotretinoin to a range of about 5 μm to about 30 μm, thereby improving the bioavailability of isotretinoin.

European Patent No. EP 0 184 942 B1, assigned to Ortho Pharmaceutical Corporation discloses pharmaceutical compositions in the form of a soft gelatin capsule having no more than a 22% wax content, as a critical limitation of the patent. Higher wax content tends to diminish the bioavailability.

U.S. Pat. No. 7,435,427, assigned to Galephar, discloses gelatin capsules comprising a semi solid suspension of isotretinoin containing at least two lipidic excipients.

Isotretinoin was initially developed and approved in 1982 for the treatment of acne. There are a number of ongoing studies regarding the use of isotretinoin for treatment of musculoskeletal and connective tissue inflammations, emphysema, ulcerating diseases and various cancers, namely treating cervical tumors in HIV positive women, the prevention of lung cancer in smokers and the prevention of skin cancer. Studies have been recently completed or ongoing regarding the role of isotretinoin (usually in combination with other drugs) in the treatment of neuroblastoma, recurrent prostrate cancer, leukemia, high-grade glioma, head and neck cancers and multiple myeloma. Isotretinoin has also been proved to be useful in the treatment of certain dermatological conditions such as gram-negative folliculitis, recalcitrant rosacea, pyoderma faciale, generalized lichen planus, psoriasis, cutaneous lupus erythematosus and acne fulminans, squamous cell carcinoma. It is also used for the treatment of cutaneous photoaging.

As is evident, many of these studies would target either pediatric or geriatric patients. Oral administration is the preferred route for children. However, children younger than 5-6 years of age have a difficulty in swallowing solid dosage forms like tablets or capsules. Thus an oral liquid is the preferred dosage form for pediatric patients.

Apart from the alterations in the pharmacodynamics and pharmacokinetics, the geriatric population suffers from a number of chronic conditions and physical limitations. Swallowing or chewing may be a problem for the elderly. For example, patients suffering from dry mouth or who are edentulous are incapable of chewing or swallowing. This makes the liquid dosage form a popular choice with the elderly.

A soft gel capsule is the only dosage form available for the oral administration of isotretinoin. However, the oral administration of solid forms such as tablets and capsules can prove difficult or even dangerous for children and the elderly who prefer to take liquid dosage forms. Further, it has been reported that for administering isotretinoin to children or infants extemporaneous liquid formulations are made by piercing/cut-opening or squeezing the contents of the capsule. These extemporaneous preparations lead to an increased dosing variability and toxicity due to the metabolism of isotretinoin (13-cis retinoic acid) to tretinoin (all trans-retinoic acid). Furthermore, the extemporaneous preparation and dispensing of isotretinoin solution is not possible in a typical hospital pharmacy set-up because of the poor solubility, longer solubilization time, safety issues in handling a teratogenic drug and photo-instability of the drug.

Neuroblastoma is an embryonic malignancy of sympathetic nervous system and occurs almost exclusively in infants and young children commonly aged between 1 and 5 years. Currently, clinical trials are going forward to study the effectiveness of isotretinoin in the treatment of neuroblastoma in children. Treatment with isotretinoin is initiated during the maintenance phase. Alternate courses of isotretinoin and an anticancer drug are given. For example, Phase II studies (sponsored by St. Jude Children's Research Hospital, NIH, AstraZeneca) on oral maintenance therapy with isotretinoin and topotecan, has been completed. These trials have been conducted using isotretinoin soft gel capsules. During these trials children were either trained to swallow the capsule or the contents from the capsule were squeezed into food causing increased dosing variability. Therefore, in the light of prior art there is an unmet need of a stable oral liquid formulation of isotretinoin.

Liquids are homogeneous preparations containing one or more active ingredients dissolved or suspended in a suitable vehicle or carrier. These include solutions, syrups, suspensions, elixirs, or concentrates. Oral liquid dosage forms offer unique advantages over the solid dosage forms like tablets and capsules. In addition to being more patient compliant, liquid dosage forms provide a more reproducible bioavailability. These dosage forms provide rapid absorption of drug from the GI tract. Additionally, liquid dosage forms allow the use of flavoring and/or palatability agents, which further promotes patient acceptance and compliance. Further, liquid formulations provide the option of a flexible dosing regime based on body weight or body surface area. The most common liquid dosage forms include suspensions and solutions.

Solutions offer several advantages over other liquid dosage forms. These are absorbed faster and generally cause less irritation of the gastrointestinal mucosa. Moreover, phase separation upon storage is not a concern with solutions. Compared with suspensions, solutions are free from the gritty-feeling that particles in a suspension might cause. The other advantages offered by the solution dosage form is that these do not need to be shaken before use, the accuracy of the dose is likely to be more than with the equivalent suspension, no particle growth is observed over time and it presents a homogeneous feel and taste. Another important advantage offered by solution is the reduction of lower inter-subject variability in pharmacokinetics, especially for highly variable molecules for e.g., isotretinoin. Further, a solution dosage form of isotretinoin has not been available.

Isotretinoin is a relatively water insoluble compound and it degrades when exposed to light and atmospheric oxygen. Further isotretinoin i.e., 13-cis retinoic acid, is a geometric isomer of tretinoin i.e., all-trans retinoic acid. These isomers show reversible interconversion. This interconversion may result in a variation in the content of active ingredient (isotretinoin) being delivered. Owing to the tendency of isotretinoin to get oxidized easily and convert into its geometric isomer, and its relative insolubility, it is difficult to formulate in a solution.

While this is known to a person skilled in the art that the drug can usually be solubilized by the addition of surfactants or co-surfactants or combination thereof to make a clear solution or micro-emulsion, the use of surfactants is associated with both bitter taste and gastric mucosal irritation.

The present invention discloses a stable pharmaceutical composition of isotretinoin in which isotretinoin is solubilized using a lipophilic carrier or a combination of lipophilic/hydrophilic carriers without the use of an additional surfactant or emulsifier. Further, this solution is substantially free of an alcoholic carrier and exhibits no bitter taste.

### SUMMARY OF THE INVENTION

In one general aspect the present invention provides for a pharmaceutical solution which includes isotretinoin or pharmaceutically acceptable salts thereof and a pharmaceutically acceptable carrier.

Embodiments of the invention may include one or more of the following features. For example, the carrier may be a lipophilic carrier or a combination of lipophilic/hydrophilic carriers. The lipophilic carrier may be fatty acid esters, fatty acids, fatty alcohols, vegetable oil or a combination thereof. The hydrophilic carrier may be monohydric alcohols, glycols, polyols, glycerols or combination thereof.

The fatty acid ester may a polyol ester of medium chain fatty acid selected from esters and mixed esters of glycerol, propylene glycol, polyglycerol and polyethylene glycol with medium chain fatty acids or mixtures thereof. The fatty acid may be C₆-C₂₀ saturated, mono, di-unsaturated acid or mixtures thereof. The fatty alcohol may be C₆-C₂₀ saturated, mono, di-unsaturated alcohol or mixtures thereof. The vegetable oil may be kernel oil, almond oil, groundnut oil, olive oil, soybean oil, safflower oil, sunflower oil, palm oil, sesame oil, canola oil or corn oil or mixtures thereof.

The isotretinoin or a salt thereof may be present in an amount of about 0.01% to about 3.0% by weight of the composition. The pharmaceutical solution may further include one or more pharmaceutically acceptable excipients selected from one or more of antioxidants, chelating agents, preservatives, colors, sweeteners or flavors or mixtures thereof.

The solution is stable during storage at 40° C.±2° C. and 75%±5% Relative Humidity; and 25° C.±2° C. and 60%±5% Relative Humidity. When administered to a human patient in a fed state, the solution exhibits a maximum plasma concentration (Cₘₐₓ) of isotretinoin comparable to that exhibited under fasting state.

In another general aspect, the present invention also provides for a process of preparing a pharmaceutical solution of isotretinoin. The process includes: (i) dissolving isotretinoin in a carrier by continuous stirring at room temperature, or at higher temperatures, till a homogenous solution is formed; and (ii) cooling the solution of step (i) to room temperature, optionally, adding one or more excipients.

In another general aspect there is provided a process of preparing a pharmaceutical solution of isotretinoin. The process includes: (i) dissolving one or more excipients in a carrier by continuous stirring; (ii) dissolving isotretinoin in the solution of step (i) by continuous stirring at room temperature, or at higher temperatures, till a homogenous solution is formed; and (iii) cooling the solution of step (ii) to room temperature.

In yet another general aspect there is provided a process of preparing a pharmaceutical solution of isotretinoin. The process includes: (i) dissolving one or more excipients in a carrier by continuous stirring; (ii) suspending the isotretinoin in the solution of step (i) under continuous stirring and milling the suspension to form a homogenous micronized suspension; (iii) diluting the suspension of step (ii) with a carrier to the desired concentration and filling in the storage container; and (iv) diluting the suspension of step (iii) with a suitable carrier to form a clear solution of isotretinoin at the time of administration.

Embodiments of the process may include one or more of the following features. For example, the pharmaceutical solution is suitable for packaging into multi-dose or unit-dose packages without producing discoloration or degradation.

In a final general aspect there is provided a method of treating acne, musculoskeletal and connective tissue inflammations, emphysema, ulcerating diseases, cervical tumors in HIV positive women, lung cancer in smokers, skin cancer, neuroblastoma, recurrent prostate cancer, leukemia, high-grade glioma, head and neck cancers, multiple myeloma, gram-negative folliculitis, recalcitrant rosacea, pyoderma faciale, generalized lichen planus, psoriasis, cutaneous lupus erythematosus and acne fulminans, squamous cell carcinoma, cutaneous photoaging and other off-label indications of isotretinoin. The method includes administering a pharmaceutical solution comprising isotretinoin or pharmaceutically acceptable salts thereof and a pharmaceutically acceptable carrier.

### DETAILED DESCRIPTION OF THE INVENTION

The present invention is directed to a pharmaceutical solution, which includes isotretinoin or salts thereof. Also provided is a process for preparing these compositions.

Further, the present invention encompasses the isotretinoin composition prepared in the form of suspension for extended stability which is diluted with the carrier at the time of administration to form a solution of isotretinoin.

The term “about” as used herein means up to plus or minus 10% of the particular term.

The term “polyol esters of medium chain fatty acids” includes esters and mixed esters of glycerol, propylene glycol, polyglycerol or other open chain polyols such as polyethylene glycol, reacted with medium chain fatty acids, wherein said acid has a chain length between 6 and 12 carbon atoms.

The term “stabilized” as used herein refers to the solution of isotretinoin which is chemically stable against oxidation and degradation. Further this solution on storage at accelerated conditions of 40° C.±2° C./75%±5% RH for 6 months, contains no more than 2.5% of total related substances, including tretinoin, 4-oxo-isotretinoin and others. Assays of the composition of the present invention during its shelf-life shows acceptable levels of isotretinoin (90-110% of label claim). The solution of the present invention contains isotretinoin in a concentration that is below its saturation solubility. Therefore, it is also physically stable and does not precipitate isotretinoin from the solution during storage at recommended storage conditions.

Isotretinoin being used in the compositions of the present invention may be present in the form of a free acid or its pharmaceutically acceptable salts, such as alkali metal salts. Isotretinoin is 13-cis retinoic acid. Tretinoin (all-trans retinoic acid) and isotretinoin are geometric isomers and show reversible interconversion in vivo. The administration of one isomer gives rise to another. This needs to be controlled and monitored adequately so that an accurate dose of the desired therapeutic agent (isotretinoin) can be administered. Other major metabolites of Isotretinoin are 4-oxo-isotretinoin and its geometrical isomer 4-oxo-tretinoin.

The carrier used in the compositions of the present invention includes one or more lipophilic carrier or a combination of lipophilic/hydrophilic carriers. The lipophilic carrier may be a single carrier or an appropriate combination of multiple miscible lipophilic carriers. Hydrophilic carrier may optionally be combined with the lipophilic carrier of the present composition to the extent that it remains miscible and forms a single phase system.

The lipophilic carrier may be including fatty acid esters, fatty acids, fatty alcohols or vegetable oil or a combination thereof.

Fatty acid esters include polyol esters of medium chain fatty acids. Polyol esters of medium chain fatty acids are selected from esters and mixed esters of glycerol, propylene glycol, polyglycerol and polyethylene glycol with medium chain fatty acids or a combination thereof. Particularly, the polyol ester of medium chain fatty acid, is a medium chain triglyceride or propylene glycol mono or diesters.

Medium chain triglycerides are medium chain (C₆ to C₁₂) fatty acid esters of glycerol and are very stable to oxidation. Examples of medium chain fatty acids include caproic acid, caprylic acid, capric acid and lauric acid. Commercially available examples of medium chain triglycerides include Neobee® 0 and Neobee® M5, Miglyol® 810, 812, 818 and 829, Captex® 350, 355 and 810D, Labrafac™ lipophile WL 1349, Crodamol™ GTCC.

Propylene glycol mono or diesters include propylene glycol monolaurate, propylene glycol monomyristate, propylene glycol dicaprylate/dicaprate or a combination thereof. Commercially available examples of propylene glycol dicaprylate or dicaprate include Miglyol® 840, Captex® 200P, Labrafac™ PG, Estol® 1526, Mazol® PG-810, and Neobee® M-20.

Specific examples of fatty acids include C₆-C₂₀ saturated or mono or di-unsaturated acid, for example, oleic acid, linoleic acid, caprylic acid or caproic acid.

Examples of fatty alcohols used in the compositions of the present invention include C₆-C₂₀ saturated or mono or di-unsaturated alcohol, for example, oleyl alcohol, capryl alcohol or capric alcohol.

Specific examples of vegetable oil used in the compositions of the present invention include kernel oil, almond oil, groundnut oil, olive oil, soybean oil, safflower oil, sunflower oil, palm oil, sesame oil, canola oil or corn oil or mixtures thereof. Particularly, the vegetable oil used in the compositions of the present invention is olive oil or soybean oil.

The hydrophilic carrier used in the composition may be selected from the group comprising monohydric alcohols, glycols, polyols or glycerols or a combination thereof. Examples of hydrophilic carriers include monohydric alcohols such as ethanol, glycols such as propylene glycol, polyethylene glycols, poly-propylene glycols, triethylene glycol; polyol such as sorbitol and glycerin.

Preferably, the carrier used in the compositions of the present invention is selected from a fatty acid ester, a vegetable oil or a suitable combination thereof. More particularly, compositions of present invention contain a fatty acid esters as the carrier.

Further, the carriers used in the composition of the present invention are characterized by their acid value, hydroxyl value, iodine value, peroxide value and saponification value.

The “acid value” may be defined as the number of mg of potassium hydroxide (KOH) required to neutralize 1 g of a sample. It has been observed that the lower the acid value, the higher the stability of the composition. The vehicle used in the composition should have an acid value less than 1, particularly less than 0.5, and more particularly less than 0.2.

“Hydroxyl Value” is a measure of hydroxyl (univalent OH) groups in an organic material. It has been observed that the lower the hydroxyl value, the higher the stability of the composition. The vehicle used in the composition should have a hydroxyl value of less than 100, particularly less than 50, and more particularly less than 10.

“Iodine Value” is a measure of the unsaturation of fats and oils and is expressed in terms of the number of centigrams of iodine absorbed per gram of sample (% iodine absorbed). It has been observed that the lower the iodine value, the higher the stability of the composition. The vehicle used in the composition should have an iodine value of less than 10, preferably less than 5, and more preferably less than 1.

“Peroxide Value” is a measure of the extent of fat or oil oxidation of a substance by measuring the amount of peroxides present. Peroxides are intermediate compounds formed during the oxidation of lipids, which may react further to form the compounds that can cause rancidity. It has been observed that the lower the peroxide value, the higher the stability of the composition. The vehicle used in the composition should have a peroxide value of less than 10, particularly less than 6, and more particularly less than 1.

“Saponification Value” is the amount of alkali necessary to saponify a definite quantity of a substance. It is commonly expressed as the number of milligrams of potassium hydroxide (KOH), or Sodium Hydroxide (NaOH), required to saponify 1 gram of the substance. It has been observed that the higher the saponification value, the higher the stability of the composition. The vehicle used in the composition should have a saponification value of higher than 200, particularly higher than 250, and more particularly higher than 300.

The pharmaceutical composition of the present invention further includes one or more pharmaceutically acceptable excipients, such as, antioxidants, chelating agents, preservatives, colors, sweeteners or flavors or mixtures thereof.

The antioxidants employed in the compositions of the present invention may include α-tocopherol, butylated hydroxyl anisole (BHA), butylated hydroxy toluene (BHT), ascorbyl palmitate and propyl gallate.

Suitable preservatives used in the compositions of the present invention include methyl paraben, ethyl paraben, propyl paraben, butyl paraben, benzoic acid, sodium benzoate, benzyl alcohol, sorbic acid and potassium sorbate.

Examples of chelating agents include, but not limited to, disodium EDTA, tartaric acid, malic acid and citric acid.

Examples of sweeteners include sorbitol, mannitol, fructose, sucrose, maltose, isomalt, glucose, hydrogenated glucose syrup, xylitol, caramel, saccharin, sodium or calcium saccharin, aspartame, acesulfame potassium, sodium cyclamate, or sucralose.

Coloring agents and flavoring agents may be selected from any FDA approved colors and flavors for oral use.

The isotretinoin solution of the present invention may further comprise one or more pharmaceutically acceptable excipients, which are soluble or miscible with the lipophilic carrier phase or an appropriate combination of the miscible lipophilic and hydrophilic phase to enhance the physical and/or chemical and/or microbiological stability of the isotretinoin solution.

The composition of the present invention may be prepared in accordance with methods well known to the person skilled in the field of pharmacy. According to one of the embodiments composition of the present invention is prepared by dissolving isotretinoin in the lipophilic carrier by continuous stirring at room temperature, or higher temperatures. This solution can also be prepared by using sonication. Additionally, an inert gas such as nitrogen may be purged through the solution during manufacturing process to protect isotretinoin against oxidation from atmospheric air or entrapped air. Since the higher temperatures during the manufacturing process are linked to higher related substances, the pharmaceutical composition of the present invention is suggested to be made at about 50° C., although higher temperatures can also be used.

Alternatively, the composition of the present invention may be prepared in a way so that the antioxidant and preservative administration to patients are reduced significantly upon administration of isotretinoin oral solution. For instance, the composition may be prepared in the form of suspension for extended stability (as it contains higher concentration of antioxidant and preservative), which may be diluted at the time of administration to form a solution having reduced concentration of the antioxidant and preservative.

According to another embodiment the isotretinoin solution may be packed in oxidation and/or light resistant packaging. The packaging may be closed and the head space may be filled with an inert gas, for example nitrogen. For multidose packages, the closures are child-resistant, yet elder friendly. Stability is ensured even after multiple openings of the packaging for dispensing the dose. Further the package may be supplied with calibrated equipment such as a dropper, medication cup, a calibrated syringe with neck adaptors to deliver an accurate dose of the drug. The package also provides for the flexible dosing of the solution while avoiding contact of the teratogenic drug with the caregiver.

According to another embodiment, the isotretinoin solution is placed in an amber colored bottle. The bottles may be made of glass or suitable plastic material, which is inert and can store isotretinoin throughout its shelf life. The liner material of closures coming in contact with the product may be made of expanded polyethylene, thin aluminium strip or other non-reactive and non-shedding material for pharmaceutically acceptable stability of composition.

The invention also relates to a method of treatment of neuroblastoma, or acne by administering an effective amount of a composition of the present invention to a patient in need of such treatment.

Further the compositions of the present invention may also be used to treat other diseases requiring administration of a retinoid such as musculoskeletal and connective tissue inflammations, emphysema, ulcerating diseases, cervical tumors in HIV positive women, prevention of lung cancer in smokers, prevention of skin cancer, recurrent prostrate cancer, leukemia, high-grade glioma, head and neck cancers, multiple myeloma, gram-negative folliculitis, recalcitrant rosacea, pyoderma faciale, generalized lichen planus, psoriasis, cutaneous lupus erythematosus and acne fulminans, squamous cell carcinoma, cutaneous photoaging and other off-label indications of Isotretinoin, where therapeutically effective amount can be provided by the pharmaceutical composition of the present invention.

The following examples represent various embodiments according to the present invention. The examples are given solely for the purpose of illustration and are not to be construed as limitations of the present invention, as many variations thereof are possible without departing from the spirit and scope of the invention.

### EXAMPLES

### Examples 1, 2, 3 and 4

### Oral Solution Containing Isotretinoin

| S. | | Example 1 | Example 2 | Example 3 | Example 4 |
|---|---|---|---|---|---|
| No | Ingredients | Percent (%) w/w (total weight of the composition) | | | |
| 1. | Isotretinoin | 0.63 | 0.63 | 0.63 | 0.11 |
| 2. | Butylated hydroxy | 0.04 | 0.05 | 0.06 | 0.05 |
| | anisole (BHA) | | | | |
| 3. | Caprylic/capric | 99.33 | 99.32 | 99.31 | 99.84 |
| | triglyceride | | | | |
| | (Miglyol ® 810) | | | | |

### Process:

1. Butylated hydroxy anisole (BHA) was dissolved in caprylic/capric triglyceride (Miglyol® 810) under continuous stirring.
2. Isotretinoin was dissolved in the solution of step 1 under stirring at 40°-45° C. (Examples 1, 2, 3) or room temperature (Example 4) to form a clear and homogenous solution.

The oral solution of examples 1, 2, 3 and 4 were subjected to stability studies at 40° C.±2° C. and 75%±5% relative humidity (RH) for a period of six months. Stability of the solution was also evaluated at 25° C.±2° C. and 60%±5% relative humidity (RH). The results are provided in Table 1(a) and Table 1(b).

TABLE 1(a)

| | Example 1 | | | Example 2 | | |
|---|---|---|---|---|---|---|
| | | After 6 | After 12 | | After 6 | After 12 |
| | | months at | months at | | months at | months at |
| | | 40° C. and | 25° C. and | | 40° C. and | 25° C. and |
| Parameters | Initial | 75% RH | 60% RH | Initial | 75% RH | 60% RH |
| Assay (% w/w) | 102.2 | 94.9 | 101.7 | 99.9 | 96.1 | 101.1 |
| BHA Content (% w/w) | 96.0 | 94.5 | 97.5 | 98.0 | 96.2 | 96.0 |
| Total RS | 0.391 | 2.380 | 1.123 | 0.339 | 2.228 | 1.439 |

TABLE 1(b)

| | Example 3 | | | Example 4 | | |
|---|---|---|---|---|---|---|
| | | After 6 | After 12 | | After 6 | After 6 |
| | | months at | months at | | months at | months at |
| | | 40° C. and | 25° C. and | | 40° C. and | 25° C. and |
| Parameters | Initial | 75% RH | 60% RH | Initial | 75% RH | 60% RH |
| Assay | 100.9 | 95.7 | 97.6 | 101.3 | 94.8 | 97.2 |
| (% w/w) | | | | | | |
| BHA Content (% w/w) | 100.5 | 99.8 | 95.0 | 101.4 | 97.6 | 99.8 |
| Total RS | 0.704 | 2.335 | 1.295 | 0.313 | 1.693 | 1.085 |

### Examples 5 and 6

### Oral Solution Containing Isotretinoin

| | | Example 5 | Example 6 |
|---|---|---|---|
| | | Percent (%) w/w | Percent (%) w/w |
| | | (total weight of the | (total weight of the |
| S. No | Ingredients | composition) | composition) |
| 1. | Isotretinoin | 0.63 | 0.63 |
| 2. | Butylated hydroxy | 0.04 | 0.05 |
| | anisole (BHA) | | |
| 3. | Caprylic/Capric | 99.33 | 99.32 |
| | triglyceride | | |
| | (Captex ® 355) | | |

### Process:

1. Butylated hydroxy anisole (BHA) was dissolved in caprylic/capric triglyceride (Captex® 355) under continuous stirring.
2. Isotretinoin was dissolved in the solution of step 1 under stirring at 40°-45° C. (Examples 5, 6) to form a clear and homogenous solution.

The oral solutions of examples 5 and 6 were subjected to stability studies at 40° C. and 75% relative humidity (RH) for the period of six months. Stability of the solution was also evaluated at 25° C.±2° C. and 60%±5% relative humidity (RH). The results are provided in Table 2.

TABLE 2

| | Example 5 | | | Example 6 | | |
|---|---|---|---|---|---|---|
| | | After 6 | After 12 | | After 6 | After 12 |
| | | months at | months at | | months at | months |
| | | 40° C. and | 25° C. and | | 40° C. and | 25° C. and |
| Parameters | Initial | 75% RH | 60% RH | Initial | 75% RH | 60% RH |
| Assay (% w/w) | 101.3 | 96.5 | 100.8 | 103.0 | 96.6 | 102.0 |
| BHA Content (% w/w) | 95.5 | 92.0 | 97.5 | 98.0 | 90.6 | 96.0 |
| Total RS | 0.384 | 1.478 | 0.730 | 0.362 | 1.480 | 0.821 |

### Examples 7 and 8

### Oral Solution Containing Isotretinoin

| | | Example 7 | Example 8 |
|---|---|---|---|
| | | Percent (%) w/w | Percent (%) w/w |
| | | (total weight of | (total weight of |
| S. No | Ingredients | the composition) | the composition) |
| 1. | Isotretinoin | 0.64 | 0.86 |
| 2. | Butylated hydroxy anisole | 0.05 | 0.05 |
| | (BHA) | | |
| 3. | Propylene glycol | 99.31 | 99.09 |
| | dicaprylate/dicaprate | | |
| | (Labrafac ™ PG) | | |

### Process:

1. Butylated hydroxy anisole (BHA) was dissolved in propylene glycol dicaprylate/dicaprate (Labrafac™ PG) under continuous stirring.
2. Isotretinoin was dissolved in the solution of step 1 by sonication to form a clear and homogenous solution.

The oral solution of Examples 7 and 8 was subjected to stability studies at 40° C. and 75% relative humidity (RH) for the period of three months. Stability of the solution was also evaluated at 25° C.±2° C. and 60%±5% relative humidity (RH). The results are provided in Table 3.

TABLE 3

| | Example 7 | | | Example 8 | | |
|---|---|---|---|---|---|---|
| | | After 3 | After 3 | | After 3 | After 3 |
| | | months at | months at | | months at | months at |
| | | 40° C. and | 25° C. and | | 40° C. and | 25° C. and |
| Parameters | Initial | 75% RH | 60% RH | Initial | 75% RH | 60% RH |
| Assay (% w/w) | 95.4 | 92.6 | 95.0 | 94.8 | 90.9 | 93.3 |
| BHA Content (% w/w) | 95.6 | 92.6 | 93.4 | 93.8 | 89.4 | 91.6 |
| Total RS | 0.646 | 1.421 | 0.969 | 0.630 | 1.381 | 0.996 |

### Example 9

### Oral Solution Containing Isotretinoin

| S. | | Percent (%) w/w (total weight |
|---|---|---|
| No | Ingredients | of the composition) |
| 1. | Isotretinoin | 0.64 |
| 2. | Butylated hydroxy anisole (BHA) | 0.05 |
| 3. | Benzoic acid | 0.025 |
| 4. | Banana flavor | 1.00 |
| 5. | Caprylic/Capric triglyceride | 98.29 |
| | (Miglyol ® 810) | |

### Process:

1. Butylated hydroxy anisole (BHA) was dissolved in caprylic/capric triglyceride (Miglyol® 810) under continuous stirring.
2. Isotretinoin was dissolved in the solution of step 1 under stirring at 40-45° C. to form a clear solution.
3. The solution of step 2 was cooled down to room-temperature and benzoic acid and banana flavor were dissolved under stirring to form homogenous solution.

The oral solution of example 9 was subjected to stability studies at 40° C. and 75% relative humidity (RH) for the period of six months. Stability of the solution was also evaluated at 25° C.±2° C. and 60%±5% relative humidity (RH). The results are provided in Table 4.

TABLE 4

| | Example 9 | | |
|---|---|---|---|
| | | After 6 months at | After 6 months at |
| | | 40° C. and 75% | 25° C. and 60% |
| Parameters | Initial | RH | RH |
| Assay (% w/w) | 101.9 | 96.8 | 99.1 |
| BHA Content (% w/v) | 100.0 | 94.0 | 96.0 |
| Preservative content | 92.0 | 88.8 | 90.0 |
| (% w/w) | | | |
| Total RS | 0.638 | 1.964 | 1.137 |

### Example 10

### Oral Solution Containing Isotretinoin

| | | Percent (%) w/w |
|---|---|---|
| | | (total weight of |
| S. No | Ingredients | the composition) |
| 1. | Isotretinoin | 1.06 |
| 2. | Butylated hydroxy anisole (BHA) | 0.05 |
| 3. | Propylene glycol monolaurate | 98.89 |

### Process:

1. Butylated hydroxy anisole (BHA) was dissolved in propylene glycol monolaurate under continuous stirring.
2. Isotretinoin was finally dissolved in the solution of step 1 under stirring at 40-45° C. to form a clear and homogenous solution.

### Example 11

### Oral Solution Containing Isotretinoin

| | | Percent (%) w/w |
|---|---|---|
| | | (total weight of |
| S. No | Ingredients | the composition) |
| 1. | Isotretinoin | 0.64 |
| 2. | Butylated hydroxy anisole (BHA) | 0.05 |
| 3. | Benzoic acid | 0.025 |
| 4. | Caprylic/Capric triglycerides | 74.47 |
| | (Miglyol ® 810) | |
| 5. | Oleic acid | 24.82 |

### Process:

1. Butylated hydroxy anisole (BHA) and benzoic acid were dissolved in the carrier combination of caprylic/capric triglycerides (Miglyol® 810) and oleic acid under continuous stirring.
2. Isotretinoin was dissolved in the solution of step 1 under continuous stirring at about 40° C. to form a clear solution.

### Example 12

### Oral Solution Containing Isotretinoin Prepared by Dilution of Isotretinoin Suspension with Oleic Acid

| | | Percent (%) w/w |
|---|---|---|
| | | (total weight of |
| S. No | Ingredients | the composition) |
| 1. | Isotretinoin | 2.55 |
| 2. | Butylated hydroxy anisole (BHA) | 0.04 |
| 3. | Benzoic acid | 0.05 |
| 4. | Caprylic/capric triglycerides | 97.44 |
| | (Miglyol ® 810) | |

### Process:

1. Butylated hydroxy anisole (BHA) and benzoic acid were dissolved in the lipophilic carrier of caprylic/capric triglycerides (Miglyol® 810) under continuous stirring.
2. Isotretinoin was suspended in the solution of step 1 under continuous stirring for about 5 min and then colloid milling done to form a homogenous micronized suspension.
3. The suspension of step 2 is diluted with the placebo solution (1:1) to make the isotretinoin suspension containing 1.277% isotretinoin. This suspension is filled in amber glass bottles with closures having EPE liner.
4. The suspension of step 3 is further diluted with oleic acid (1:1) to form a clear solution containing 0.64% isotretinoin just before administration which results in reducing the butylated hydroxyl anisole and benzoic acid content to 0.02% and 0.025% from the initial level of 0.04% and 0.05%, respectively.

### Example 13

### Bioavailability Studies

### (I) Comparison of Oral Bioavailability of Solution of Isotretinoin Relative to Capsule Formulation in Healthy Adult Male Human Subjects Under Fed State

Comparative bioavailability studies of isotretinoin 6 mg/ml solution (dose 5 ml) (Examples 2 and 6) relative to isotretinoin soft-gel capsules 30 mg (containing 30 mg of isotretinoin) were conducted in healthy adult male human subjects under fed state because the isotretinoin soft-gel capsules are indicated for use under fed state in the product pack insert. Pharmacokinetic parameters for isotretinoin solution and capsule were compared. Results from these studies confirmed that the extent of absorption from the isotretinoin solution was greater by approximately 20% when compared to conventional marketed capsule formulation under fed state.

Comparative pharmacokinetic parameters of two formulations after single oral dose are depicted in Table 5 (solution of Example 2) and Table 6 (solution of Example 6)

TABLE 5

| | Solution | | Capsule | | Relative |
|---|---|---|---|---|---|
| Parameters | formulation | | formulation | | bioavailability |
| AUC₀₋₉₅ (ng/ml · hr) | 6920.46 | (17.4) | 5752.91 | (38.1) | 20.29 |
| AUC_{0-∞} (ng/ml · hr) | 7202.44 | (18.4) | 6305.32 | (37.0) | 14.23 |
| Cₘₐₓ (ng/ml) | 591.68 | (32.0) | 516.26 | (55.0) | 14.61 |

TABLE 6

| | Solution | | Capsule | | formulation |
|---|---|---|---|---|---|
| Parameters | formulation | | bioavailability | | Relative |
| AUC₀₋₉₅ (ng/ml · hr) | 6361.50 | (22.7) | 5449.16 | (37.2) | 16.74 |
| AUC_{0-∞} (ng/ml · hr) | 6603.10 | (22.2) | 5696.48 | (35.3) | 15.92 |
| Cₘₐₓ (ng/ml) | 521.30 | (31.7) | 500.13 | (37.3) | 4.23 |

### (II) Comparison of Oral Bioavailability of Oral Solution of Isotretinoin Under Fed and Fasting Conditions

Comparative bioavailability study of isotretinoin solution was conducted in healthy adult male human subjects under fed and fasting conditions. It has been reported in the product pack insert of Isotretinoin soft-gel capsules (Accutane®, Roche, USA) that both the peak plasma concentration (Cₘₐₓ) and the total exposure (AUC) of isotretinoin were more than doubled following a standardized high-fat meal when compared with Accutane® given under fasted conditions. Significantly reduced food-effect has been observed on the extent of absorption (AUC) when isotretinoin is given as solution with food with almost no effect observed on the rate of absorption, i.e. Cₘₐₓ.

Comparative pharmacokinetic parameters under fed and fasting conditions are depicted in Table 7.

TABLE 7

| | Least squares | Least squares | Food effect in % |
|---|---|---|---|
| Parameters | mean (fasting) | mean (fed) | [(Fed-Fasting)/Fed] |
| Cₘₐₓ | 748.40 | 762.02 | +1.78% |
| AUC₀₋ₜ | 8984.35 | 13297.53 | +32.43% |
| AUC_{0-∞} | 9514.38 | 14082.15 | +32.44% |

While several particular forms of the invention have been illustrated and described, it will be apparent that various modifications and combinations of the invention detailed in the text can be made without departing from the spirit and scope of the invention. Accordingly, it is not intended that the invention be limited, except as by the appended claims.

1. A stable liquid solution for an oral administration comprising isotretinoin, an antioxidant and a lipophilic carrier wherein said composition contains related substance not more than 2.5% w/w when stored for 6 months at a temperature 40° C.±2° C. and RH of 75%±5%.

2. The stable liquid solution for an oral administration according to claim 1 wherein the isotretinoin dose is about 30 mg per 5 mL of the composition.

3. The stable liquid solution for an oral administration according to claim 1, wherein the antioxidant are selected from the group consisting of α-tocopherol, butylated hydroxyl anisole, butylated hydroxy toluene, ascorbyl palmitate and propyl gallate.

4. The stable liquid solution for an oral administration according to claim 1, further comprising one or more pharmaceutically acceptable excipients selected from one or more of chelating agents, preservatives, colors, sweeteners or flavors or mixtures thereof.

5. The stable liquid solution for an oral administration according to claim 1, wherein the lipophilic carrier comprises fatty acid esters, fatty acids, fatty alcohols, vegetable oil or a combination thereof.

6. The stable liquid solution for an oral administration according to claim 1, wherein the isotretinoin or a salt thereof is present in an amount of about 0.01% to about 3.0% by weight of the composition.

7. The stable liquid solution for an oral administration according to claim 1, wherein the liquid solution when administered to a human patient in a fed state, exhibits a maximum plasma concentration (Cmax) of isotretinoin comparable to that exhibited under fasting state.

8. The stable liquid solution for an oral administration according to claim 1 wherein the process of preparing a stable liquid solution of isotretinoin, comprises the steps of: (i) dissolving an antioxidant in a lipophilic carrier by continuous stirring; (ii) dissolving isotretinoin in the solution of step (i) by continuous stirring at room temperature, or at higher temperatures, till a homogenous solution is formed; and (iii) cooling the solution of step (ii) to room temperature.

9. The stable liquid solution for an oral administration according to claim 1, wherein the liquid solution is packed in multi-dose or unit-dose packages.

10. The stable liquid solution for an oral administration according to claim 1, wherein the extent of absorption of 5 mL of said solution is about 20% greater than that provided by the commercially available daily dose of a capsule of 30 mg of isotretinoin when ingested orally.

11. The stable liquid solution for an oral administration according to claim 1, wherein oral administration of the liquid solution exhibits minimal food effect on Cₘₐₓ.

12. A stable liquid solution for an oral administration comprising isotretinoin or pharmaceutically acceptable salts thereof, an antioxidant and a lipophilic carrier comprising a combination of medium chain fatty acid ester of glycerol and oleic acid.

13. The stable liquid solution for an oral administration according to claim 12, wherein a ratio of the medium chain fatty acid ester of glycerol to the oleic acid is about 3:1.

14. The stable liquid solution for an oral administration according to claim 13, wherein the medium chain fatty acid ester of glycerol comprises caprylic/capric triglycerides.
